(19) United States
(12) Patent Application Publication
(10) Pub. No.: US 2025/0065138 A1
(43) Pub. Date: Feb. 27, 2025

(54) SYSTEMS METHODS OF SAFELY DELIVERING AN EFFICIENT AMOUNT OF OXYGEN TO ESSENTIAL ORGANS DURING CARDIOPULMONARY RESUSCITATION

(71) Applicant: Noam GAVRIELY, Haifa (IL)

(72) Inventor: Noam GAVRIELY, Haifa (IL)

(21) Appl. No.: 18/727,669

(22) PCT Filed: Jan. 10, 2023
(86) PCT No.: PCT/IB2023/050197

§ 371 Date: Jul. 9, 2024

Related U.S. Application Data
(60) Provisional application No. 63/298,297, filed on Jan. 11, 2022.

(51) Int. Cl. A61N 1/39; A61B 17/132; A61H 9/00; A61H 31/00; A61M 1/16; A61M 16/00; A61M 16/04; A61M 16/10; A61M 16/12; A61M 16/20

(52) U.S. Cl. CPC A61N 1/39044; A61B 17/1325; A61H 9/0092; A61H 31/006; A61M 16/0003; A61M 16/024; A61M 16/0434; A61M 16/1005; A61M 16/12; A61M 16/208; A61H 2201/10; A61H 2201/107; A61H 2230/206; A61H 2230/208; A61H 2230/405; A61M 1/1698; A61M 2016/0027; A61M 2202/02; A61M 2202/0225; A61M 2202/0291; A61M 2205/3344; A61M 2230/202; A61M 2230/205

(57) ABSTRACT

A system for safely delivering an efficient amount of oxygen to essential organs, during cardiopulmonary resuscitation (CPR) is described; a respective method and an endotracheal device for delivering a semi-spontaneous positive-pressure ventilation are further described; the system comprises; at least one limb compression device, a positive-pressure ventilation system, an endotracheal tube, a cardiac stimulation device, an intra-tracheal pressure sensor, a synchronizer; the method comprises; compressing at least one limb device and occluding a blood flow into the limb, delivering a mixture of gases, providing an endotracheal tube, conferring a deployed configuration, conferring a withheld configuration, performing a cardiac stimulation device, determining a pressure, synchronizing a timing; the endotracheal device comprises; an elongated tube, a sealing cuff assuming a deployed configuration and withheld

### TECHNICAL FIELD

This invention generally involves the fields of emergency medicine and cardiology and more specifically on means to improve the meaningful outcome of cardiac arrest.

### INTRODUCTION

Cardiac arrest is the cause of death of more than 500,000 people every year in the United States and of millions globally. The cause of cardiac arrest in the majority of patients is critical narrowing or blocking of coronary arteries, leading to cessation of cardiac pumping. This results in abrupt stopping of blood flow and termination of Oxygen supply to the tissues. The consequences are catastrophic. In some tissues there are reserves of Oxygen and stores of high-energy compounds and they can survive for several hours without sustaining irreversible damage (e.g. skin, fat, muscles), while other tissues (e.g. heart muscle and brain) have no such reserves and require continuous high O2 supply and therefore cannot sustain even a short period of ischemia without suffering irreversible injury and damage to the cells.

The current American Heart Association (AHA) protocol for attempting to save the cardiac arrest patient life is by administering cardio-pulmonary resuscitation, namely external chest compression and electric defibrillation in an attempt to restore the mechanical and electrical cardiac functionality also known as return of spontaneous circulation (ROSC).

The key to such jump-starting of the arrested heart is rapid restoration of blood flow to the heart muscle through the coronary arteries. However, it is clearly understood today that ROSC is not enough and avoiding neurological damage during the process of CPR is critical in order to achieve meaningful post-cardiac arrest survival. This means that blood flow and O2 supply to the brain must also be maintained during CPR. Unfortunately, the overall clinical experience with CPR after 60 years since external chest compression was first introduced is very disappointing. In a recent study CPR was performed on 8000 patient who had witnessed cardiac arrest out of the hospital. Half of them were also treated with IV epinephrine (Epi) and the other half had the same CPR protocol but without epinephrine administration. In the Epi group of the patients had ROSC upon hospital arrival, which is much higher than the ROSC in the patients not treated with Epi. However, there was no statistically significant difference between the groups when neurologically intact survival at 30 days post arrest was compared.

It is therefore clear that the use of Epi is beneficial in reviving the heartbeat, its use does not help the viability of the brain, and perhaps even hurts it. Similar results were shown in multiple clinical and experimental studies. The flow diagram of FIG. 1 shows the sequence of physiological events that occur during cardiac arrest and CPR. It is obvious that additional and better means are necessary in order to improve the meaningful outcome of CPR.

Analysis of the Current Practices and Mechanisms During CPR that Contribute to its Poor Neurological Outcome:

The administration of Epi and other vasopressors constrict the arteries, including the cerebral arteries resulting in increased resistance to flow. While this may increase blood pressure, it clearly impedes its flow and thereby the O2 transport to the brain tissue.

Immediately after cardiac arrest two vasodilating mechanisms take place in all the arteries: (a) the physiological mechanism of “Reactive Hyperemia” also known as hypoxic vasodilatation cause maximal widening of the arterioles throughout; and (b) the activity of the sympathetic nervous system stops which also contribute to widespread vasodilatation. As such when Epi and other pressor drugs are injected into a vein, they slowly move through the widely open arteries. The timing of arrival of intravenously injected Epi and other vasopressors to the body tissues is therefore highly uneven during CPR. Tissues that are nearer to the heart, such as the brain, receive the drugs first and those who are further away such as the legs get the drugs significantly later. The result is that the brain circulation constricts earlier than the peripheral circulation. By nature of simple hydrodynamics, the blood flow generated by chest compression CPR is channeled preferentially to the wide-open periphery while its flow into the constricted brain arteries is impeded. Epi arrives only later to the periphery, but due to its short half-life, only 4 minutes and the sluggish blood flow by CPR, it is already partially inactivated upon peripheral arrival. This paradoxical “steal phenomenon” deprives the brain tissue from the essential transport of Oxygen to its cells.

Positive pressure ventilation is of course necessary if the patient is not breathing. However, there are three major problems associated with uncontrolled artificial ventilation as it is currently performed: (a) too much ventilation relative to the much-reduced metabolic production of CO2 during CPR result in reduction of arterial PCO2 (PaCO2). Low PaCO2 is a potent cause of cerebral vasoconstriction, again impeding cerebral blood flow; and (b) low PaCO2 causes a shift to the left of the Oxygen-hemoglobin dissociation curve (Bohr's effect). This shift results in stronger bonding of the Oxygen to the hemoglobin and reduced off-loading of O2 molecules at the tissue. The outcome is that for each ml of blood flowing through the capillaries, much less (e.g. 20-35% less) Oxygen is delivered for any level of capillary PO2. Attempt by the tissue to extract more Oxygen from the blood is not helpful because this requires lowering the end-capillary PO2 thereby reducing the diffusional driving force that bring O2 molecules from the capillaries to the mitochondria. Impeded flow, reduced O2 delivery and smaller partial pressure gradients combine to critical limitation of Oxygen transport to the brain. Clearly hyperventilation is to be absolutely avoided during CPR as recognized by multiple animal and clinical studies. (c) positive pressure ventilation with relatively large tidal volumes increase the intra-thoracic pressures and distend the alveoli, thereby impeding the return of venous blood to the right side of the heart, compressing the pulmonary capillaries and increasing their resistance to flow and also stretching (elongating) the pulmonary capillaries, which also increase their resistance to flow. These phenomena result in filling up of the heart chambers so that, when compressed, only a small amount of blood is ejected for each compression. Note that the attempt to counteract these effects of positive pressure ventilation by applying negative pressure to the airways by maintaining the reduced chest volume due to chest compression by preventing inflow of air into the airways (e.g. by the “Impedance Threshold Device”) and by using a compression-decompression (suction) forces on the chest wall during CPR have limited benefit due to the dynamic collapse nature of the central veins and the pulmonary blood vessels as well as the distal, non-cartilaginous airways of the lung. In certain circumstances, if the trans-mural pressure on the thoracic vessels or the small airways become negative, the conduits may collapse, become flow-limited, or experience flutter.

The reduced PaCO2 associated with relatively excessive ventilation is followed by increase in pH (“Respiratory Alkalosis”), an independent cause of leftward shift of the O2-hemoglobin dissociation curve. This is further insulted by a practice, used for many years, and now discouraged, to administer IV doses of Na-bicarbonate, presumably to counteract the metabolic acidosis caused by the generation of lactic acid through anaerobic metabolism in the tissues. Bicarbonate administration further shifts the O2 dissociation curve to the left with the ensuing limitation of handing O2 molecules from the blood to the tissues.

The diagrams shown in the Drawings outline a summary of these mechanisms.

### BACKGROUND ART

The use of vasodilating drugs during CPR has been previously shown to be beneficial in multiple animal experiments by significantly increasing cerebral blood flow. The compound that was previously used is intravenous (IV) Na-Nitroprusside (SNP), a known potent vasodilator. This animal studies also revealed a drop in systemic blood pressure when SNP is administered, which is to be expected when vaso-dilatation is induced. The same drop in blood pressure is known to occur when PaCO2 is elevated.

Attempts to counteract the extreme vasodilatation described in item #2 above by mechanical methods have largely failed so far as outlined below.

Studies using the Military (Medical) Anti Shock Trousers (MAST) which is a garment with inflatable bladders that covers the entire lower body, including the abdomen and legs showed no beneficial effects. There are 3 main reasons why this method failed:

The MAST does not inflate sequentially from distal to proximal. Thereby it does not squeeze the blood from the periphery to the core. In fact, if the proximal bladders inflate before the distal ones, the MAST may act as a venous tourniquet and blood may be trapped in the periphery instead of being auto-transfused.

The MAST cannot be removed gradually. Its removal, either by deflating the bladders or by opening the Velcro® fasteners abruptly opens the peripheral vascular bed, leading to sudden drop in peripheral resistance and blood pressure, which often brings about re-collapse of the patient's homeostasis.

The compression of the abdomen by the abdominal bladder shifts the viscera caudally and limits the expansion of the lower ribs thereby causing a significant interference with respiration.

Applying the MAST takes too much time (>5 minutes). Researchers have attempted to increase peripheral resistance by tight abdominal binding during CPR. This method also failed to improve the outcome during experimental CPR in laboratory animals (pigs).

Elevation of the legs during CPR has been tried previously. However, only 45% of the blood is emptied from the legs by elevation (Blond et Al, Acta Ortho Scand. 2001). In addition, with each compression of the chest, blood does flow into the dilated arteries of the legs thereby minimizing the benefit of leg raising which requires extra personnel during transport.

Binding of the legs with Esmarch bandages was described in 1951 by Dr. Woolworth from Tasmania with beneficial effects of re-expanding the heart during open-chest cardiac massage in a child who underwent ether anesthesia for an orthopedic surgery and experienced cardiac arrest. The child did not survive.

Applying tourniquets such as used to stop bleeding in trauma or pneumatic tourniquets is a known art that could theoretically increase vascular resistance and improve the efficacy of cardiac massage by channeling the stroke volume to the essential organs. However, as is well known in orthopedics, stopping the blood flow in a limb without first fully emptying the blood is often associated with intra-vascular clotting. These clots rapidly migrate to the lungs and to the brain (Sulek 1999) upon release of the tourniquet with consequential pulmonary embolization and cerebral infarcts.

Blocking the aorta by an inflated balloon inserted via a femoral artery is a heroic measure with significant beneficial effects in experimental animals (Sesma et Al, Am. J. Emerg. Med. Effect of Intra-aortic occlusion balloon in external thoracic compressions during CPR in pigs—ScienceDirect), https://www.sciencedirect.com/science/article/abs/pii/S0735675702000402.

An important prior art is the use of Extra-Corporeal Membrane Oxygenator (ECMO) during CPR in an attempt to provide oxygenated blood to the essential organs (Speidl 2015 Extracorporeal membrane oxygenation in cardiac arrest). ECMO is often successful when used (4-54% meaningful survival), but is seldom used due to logistical difficulties, availability of teams and device and cost. It is only used in 1-2% of cardiac arrest patients in large European urban environments, https://www.escardio.org/static-Die/Escardio/Congresses/Congress management/Acute Cardiovascular Care/Documents/Slides_FP344.pdf.

### SUMMARY OF THE INVENTION

The following summary of the invention is provided to exhibit the basic understanding of some principles, underlying various aspects and features of the invention. This summary is not an extensive overview of the invention and as such it is not necessarily intended to particularly identify all key or critical elements of the invention and is not to delineate the scope of the invention. Its sole purpose is to present some concepts of the invention in a simplified form as a prelude to the following more detailed.

The invention was made in view of the deficiencies of the prior art and provides systems, methods and processes for overcoming these deficiencies. According to some embodiments and aspects of the present invention, there is provided a system for safely delivering an efficient amount of oxygen to essential organs, during cardiopulmonary resuscitation (CPR) comprises: at least one limb compression device, configured for exerting a distal-to-proximal sequential compression force onto a limb and for occluding a blood flow into the limb; a positive-pressure ventilation system, configured for delivering a mixture of gases by positive pressure. According to some embodiments and aspects of the present invention, the positive-pressure ventilation system comprises: a carbon dioxide reservoir containing a carbon dioxide enriched gas; a molecular oxygen reservoir containing a molecular oxygen enriched gas; a controllable mixing module, operationally connected to the carbon dioxide reservoir and the molecular oxygen reservoir, configured to controllably mix the molecular oxygen enriched gas with the carbon dioxide enriched gas; at least one carbon dioxide partial pressure sensor selected from the group consisting of: an arterial blood carbon dioxide partial pressure sensor and end-tidal exhaled air carbon dioxide partial pressure sensor, configured to detect a partial pressure of carbon dioxide in an arterial blood; a controller, operationally connected to the controllable mixing module and at least one carbon dioxide partial pressure sensor, configured for controlling at least one ratio selected from the group consisting of: a ratio of the molecular oxygen enriched gas and with ratio of the carbon dioxide enriched gas, in a mixture of the molecular oxygen enriched gas and the carbon dioxide enriched gas; an endotracheal tube comprising a sealing cuff disposed at a distal portion of the endotracheal tube, configured for iteratively assuming: a deployed configuration, in which the sealing cuff is engaged to an interior surface of a trachea, whilst sustaining an inflow of gases from the endotracheal tube, into the trachea, and a withheld configuration, in which the sealing cuff is disengaged from the interior surface of the trachea, whilst sustaining a spontaneous outflow of the gases from the trachea.

According to some embodiments and aspects of the present invention, the system comprises: a cardiac stimulation device, configured for returning a spontaneous circulation of the arterial blood, by providing at least one stimulation to a cardiac muscle, selected from the group consisting of: a mechanical stimulation and electrical stimulation, at a predetermined time intervals; an intra-tracheal pressure sensor configured for continuously determining a pressure inside the trachea; a synchronizer configured for timing an injection phase of the positive-pressure ventilation system with an onset of a decompression phase of the cardiac stimulation.

In some embodiments, the distal-to-proximal sequential compression force onto the limb is achieve by an up-rolling constricting elastic ring.

In some embodiments, the distal-to-proximal sequential compression force onto the limb is achieved by applying at least one element selected from the group consisting of: an elastic bandage, an elastic limb wrap with adjustable closures, an inflatable limb wrap with adjustable closures.

In some embodiments, at least one limb compression device is configured for occluding the arterial inflow of blood into the limb by applying a surface skin pressure range selected from the group consisting of: 100- and 200-mm Hg, 200- and 300 mm Hg.

In some embodiments, the mixture of gases is selected from the group consisting of: 95% molecular oxygen and 5% carbon dioxide, 0.1 to 2.0% carbon dioxide with the balance being molecular oxygen, 2.1- to 4.0% carbon dioxide with the balance being molecular oxygen, 4.1 to 5.6% carbon dioxide with the balance being molecular oxygen, 0.1 to 5.0% carbon dioxide with 30 to 50% molecular oxygen and the balance being a chemical element Xenon, 0.1 to 5.0% carbon dioxide with 30 to 50% molecular oxygen and the balance being chemical element Argon.

In some embodiments, the controllable mixing module controllably mixes carbogen gas of 5% carbon dioxide and 95% molecular oxygen with pure 100% molecular oxygen according to feedback from at least one carbon dioxide partial pressure sensor selected from the group consisting of: the arterial blood carbon dioxide partial pressure sensor and the end-tidal exhaled air carbon dioxide partial pressure sensor, to maintain the arterial blood carbon dioxide partial pressure level at 41-45 mm Hg.

In some embodiments, the controllable mixing module controllably mixes carbogen gas of 5% carbon dioxide and 95% molecular oxygen with pure 100% molecular oxygen according to feedback from at least one carbon dioxide partial pressure sensor selected from the group consisting of: the arterial blood carbon dioxide partial pressure sensor and end-tidal exhaled air carbon dioxide partial pressure sensor, to maintain the arterial blood carbon dioxide partial pressure level at at least one pressure range selected from the group consisting of 41-45 mm Hg, 46-50 mm Hg, 51-55 mm Hg, 56-65 mm Hg.

In some embodiments, the controllable mixing module controllably mixes carbogen gas of 5% carbon dioxide and 30% molecular oxygen and 65% of chemical element Xenon with a gas mixture of 30% molecular oxygen and 70% of chemical element Xenon according to feedback from at least one carbon dioxide partial pressure sensor selected from the group consisting of: the arterial blood carbon dioxide partial pressure sensor and end-tidal exhaled air carbon dioxide partial pressure sensor, to maintain the arterial blood carbon dioxide partial pressure level at 41-65 mm Hg.

In some embodiments, the controllable mixing module controllably mixes carbogen gas of 5% carbon dioxide and 50% molecular oxygen and 45% of chemical element Xenon with a gas mixture of 50% molecular oxygen and 50% of chemical element Xenon according to feedback from at least one carbon dioxide partial pressure sensor selected from the group consisting of: the arterial blood carbon dioxide partial pressure sensor and end-tidal exhaled air carbon dioxide partial pressure sensor, to maintain the arterial blood carbon dioxide partial pressure level at 41-65 mm Hg.

In some embodiments, the controllable mixing module controllably mixes carbogen gas of 5% carbon dioxide and 30% molecular oxygen and 65% of chemical element Argon with a gas mixture of 30% molecular oxygen and 70% of chemical element Argon according to feedback from at least one carbon dioxide partial pressure sensor selected from the group consisting of: the arterial blood carbon dioxide partial pressure sensor and end-tidal exhaled air carbon dioxide partial pressure sensor, to maintain the arterial blood carbon dioxide partial pressure level at 41-65 mm Hg.

In some embodiments, the controllable mixing module controllably mixes carbogen gas of 5% carbon dioxide and 50% molecular oxygen and 45% of chemical element Argon with a gas mixture of 50% molecular oxygen and 50% of chemical element Argon according to feedback from at least one carbon dioxide partial pressure sensor selected from the group consisting of: the arterial blood carbon dioxide partial pressure sensor and end-tidal exhaled air carbon dioxide partial pressure sensor, to maintain the arterial blood carbon dioxide partial pressure level at 41-65 mm Hg.

According to some embodiments and aspects of the present invention, there is provided a method of safely delivering an efficient amount of oxygen to essential organs, during cardiopulmonary resuscitation (CPR), comprises: compressing at least one limb device and occluding a blood flow into the limb, by exerting a distal-to-proximal sequential compression force onto a limb; delivering a mixture of gases by a positive-pressure ventilation comprising: providing a carbon dioxide enriched gas; providing a molecular oxygen enriched gas; controllably mixing the molecular oxygen enriched gas with the carbon dioxide enriched gas; detecting a partial pressure of carbon dioxide in an arterial blood; controlling at least one ratio selected from the group consisting of: a ratio of the molecular oxygen enriched gas and with ratio of the carbon dioxide enriched gas, in a mixture of the molecular oxygen enriched gas and the carbon dioxide enriched gas.

According to some embodiments and aspects of the present invention, the method comprises: providing an endotracheal tube comprising a sealing cuff disposed at a distal portion of the endotracheal tube; conferring to the sealing cuff a deployed configuration, in which the sealing cuff is engaged to an interior surface of a trachea, whilst sustaining an inflow of gases from the endotracheal tube, into the trachea; conferring to the sealing cuff a withheld configuration, in which the sealing cuff is disengaged from the interior surface of the trachea, whilst sustaining a spontaneous outflow of the gases from the trachea; performing a cardiac stimulation device, for returning a spontaneous circulation of the arterial blood, by providing at least one stimulation to a cardiac muscle, selected from the group consisting of: a mechanical stimulation and electrical stimulation, at a predetermined time intervals; continuously determining a pressure inside the trachea; synchronizing a timing of an injection phase of the positive-pressure ventilation system with an onset of a decompression phase of the cardiac stimulation.

According to some embodiments and aspects of the present invention, there is provided an endotracheal device for delivering a semi-spontaneous positive-pressure ventilation comprises: an elongated tube configured for endotracheal deployment; a sealing cuff disposed at a distal portion of the elongated tube, configured for iteratively assuming a deployed configuration and withheld configuration; in the deployed configuration, the sealing cuff is sprawled out, so as to engage to an interior surface of a trachea, whilst sustaining an inflow of gases from the endotracheal tube, into the trachea; in the withheld configuration, the sealing cuff is folded, so as to disengage from the interior surface of the trachea, whilst sustaining a spontaneous outflow of the gases from the trachea.

In some embodiments, the sealing cuff comprises an inflatable toroidal structure, comprising an inflatable interior lumen.

In some embodiments, the endotracheal device for delivering a semi-spontaneous positive-pressure ventilation further comprises at least one conduit connecting the inflatable interior lumen of the sealing cuff with an interior lumen of the elongated tube.

In some embodiments, the endotracheal device for delivering a semi-spontaneous positive-pressure ventilation further comprises at least one outlet on an anterior distal portion of the toroidal structure of the sealing cuff, configured to sustain an inflow of gases from the inflatable interior lumen of the sealing cuff into the trachea.

In some embodiments, the elongated tube comprises a unidirectional flow check-valve, configured to sustain an inflow of gases from the endotracheal tube, into the trachea.

The invention discloses prescribing a target hypercapnic (i.e. above normal) level of PaCO2; monitoring the PCO2 in the arterial blood (PaCO2) either by measuring it directly or by monitoring the CO2 fraction at the end of exhalation also known as End-Tidal PCO2 (PETCO2); using the PaCO2 as feedback information changing the mixing ratio of two gases, one containing 5% or 5.6% CO2 with Oxygen and, if desired, neuro-protective inert gases such as Xenon or Argon and the other with same composition but without CO2. The mixing device can increase or decrease the proportion of CO2 in the mixture and do so according to the PaCO2 (or PETCO2) instantaneous level. The prescribed hypercapnic level of PaCO2 is typically a value between 41 mm Hg and 60 mm Hg and more commonly between 46- and 55 mm Hg. These PaCO2 levels are known to cause: (a) vasodilation; (b) shift to the right of the O2-hemoglobin dissociation curve; and (c) stimulate breathing activity should the patient be able to return to spontaneous breathing.

As such, in order to counteract the systemic vasodilation caused by administering CO2-rich gas to elevate PaCO2, this invention dictates the simultaneous use of means to constrict the blood vessels in the periphery and in particular in the limbs. The invention therefore discloses application of a distal-to-proximal sequential limb-compression devices that squeeze the blood from the limbs to the central circulation and then block the re-entry of the blood into the limb. Such devices consist, but are not limited to elastic constricting rings, elastic constricting ring with constricting elastic sleeve (stockinet), manual elastic wrap, manual elastic bandage and a sequentially inflatable pneumatic constricting wrap as shown in the drawings and the detailed description of the invention.

Elevating PaCO2 causes global vasodilation therefore increasing vascular resistance in the periphery is key to channeling more of the cardiac output generated by CPR to the essential organs and in particular to the brain circulation. The second benefit of elevating PaCO2, which does not exist with SNP is the shift to the right of the O2 dissociation curve which facilitates higher off-load of O2 when the blood flows through the tissues.

Another aspect of the current invention is the means of controlling PaCO2 at a desired level by servo-control of a mixing device that mixes two gases; one containing 5 or 5.6% CO2 and the other does not. If, for example, PaCO2 or PETCO2 are lower than the prescribed value, the mixer adjusts to supply a higher Fraction of CO2 (FCO2). The mixing device-monitor feedback mechanism contains means to slow down changes of FCO2 to avoid an under-dumped feedback loop which can result in undesired oscillations of PaCO2. Since FCO2 is not the only parameter than determines PaCO2, we use Equation 1 to show all the parameters and their interactions:

$${F CO}_{2} = \frac{{Fa CO}_{2} \cdot f \cdot ( VT - V D ) - \dot{V} {CO}_{2}}{f \cdot VT} \qquad Equation 1$$

where:
FCO 2 is the delivered fraction of CO2 needed to maintain the desired level of CO2 in the blood;
PaCO2 (or PETCO2) is the desired partial pressure of CO2 in the arterial blood f is the respiratory rate;
VT is the tidal volume;
VD is the lung dead-space. It is usually taken as 2.2× body weight in Kg (or as body weight in Lbs;
\dot{V}CO₂ is the rate of metabolic production of CO2 in the tissues.

FIG. 1a shows the graphic relationships between FCO2 and for several levels of ventilation (i.e. f and VT) and PaCO2:\dot{V}CO₂.

In order to further evaluate the sensitivity and safety of the novel supply of hypercapnic gas mixture it is necessary to verify that sufficient O2 is supplied. Equation 2 calculates the effect of using this gas mixture on PaO2, the partial pressure of Oxygen in the arterial blood.

$$\dot{V} {O}_{2} = \frac{\dot{V} {CO}_{2}}{R Q} = ( {FI O}_{2} - {FAlv O}_{2} ) \cdot f \cdot ( VT - V D ) \qquad Equation 2$$

where:
\dot{V}O₂ is the metabolic O2 consumption by the tissues;
RQ is the respiratory quotient which is the ratio between CO2 production and O2 consumption. Its value is approximately 0.8 in people who consume balanced diet;
FIO₂ is the fraction of O2 in the inspired gas;
and is the Alveolar fraction of O₂. FAlvO₂.

In order to maintain near 100% saturation of the hemoglobin (e.g. 98%) in the arterial blood, even when the O2-hemoglobin dissociation curve is very shifted to the right, namely with P50=40 mm Hg (P50 is the partial pressure of oxygen at which the hemoglobin is 50% saturated), we must keep PaO2 and PAIvO2 at a higher than atmospheric level by enriching the gas mixture with Oxygen. Equation 3 calculates the needed PaO2 for 98% saturation by using the Hill Equation.

$${O}_{2} Sat = 98 \% = \frac{P O {2}^{2.8}}{P O {2}^{2.8} + P {50}^{2.8}} \qquad Equation 3$$

And after moving elements of Equation 3 around we get Equation 4:

$$P O 2 = \frac{O 2 Sat \cdot P {50}^{2.8}}{1 - O 2 Sat}$$

for O2Sat=0.98 and P50=40 we get PO2=160.6 mm Hg.

Returning to Equation 2, we can evaluate the minimum values of FIO2, f and VT for given values of Vdot CO2 and VD. As such, an important aspect of this invention is the Gas Exchange Calculator which helps determine the needed FICO2 needed in order to maintain PaCO2 for various values of tissue CO2 production. Intuitively, the lesser CO2 metabolic tissue production, the higher FICO2 must be in order to maintain PaCO2 at the desired level. Theoretically this could be done by reducing alveolar ventilation (e.g. by reducing respiratory rate or tidal volume), but this could lead to restricted Oxygen delivery, resulting in too low PaO2 and O2Sat as shown in Eq 3 and 4. As such, the Gas Exchange Calculator sets the limits of minimal alveolar ventilation that is safe for the patient's tissue oxygenation for any remaining level of metabolic rate.

Additional aspect of the invention is the supply of the servo-controlled gas mixture disclosed above not only for respiration, but also into an artificial lung or heart-lung machine such as an ECMO device or a bubble oxygenator.

A further aspect of the invention is maintaining the artificial ventilation of the patient's lung at very low or even negative pressure. This means keeping the mean airway pressure low and minimizing the rise of pressure during inspiration. Doing so reduces the impediment of the distention of the lung and the chest on venous return of blood from the periphery to the heart and the resistance to blood flow through the pulmonary capillaries. This invention discloses means of doing so by synchronizing the inspiratory (lung inflation) phase of the breathing cycle to the decompression or recoil phase of the CPR chest compression, using a method of intra-tracheal catheter ventilation and assisting exhalation by applying phased negative pressure.

This novel CPR-specific method of optimized artificial ventilation is an integral part of this invention by focusing on preventing the interference of the ventilation with pulmonary blood flow. A preferred embodiment of the ventilation component consists of the following steps that, when applied together, facilitate venous return to the right heart and filling of the left heart during the “diastole” or decompression phase of the chest compression CPR.

Synchronization of the air injection (inspiration) phase of the ventilation with the decompression part of the external cardiac massage. Doing so facilitates air entry at low driving pressure. A preferred embodiment of this synchronization is by using a high frequency-response intra-tracheal pressure sensor that feeds into a processor that determines the correct timing of the inspiratory air inflation into the lung to be at the onset of the decompression phase of CPR.

Markedly reducing the anatomical dead space so that the alveolar ventilation is maintained by a smaller tidal volume. This is done by ventilating the patient with a narrow tracheal catheter that injects the inspiratory air at the main carina. While the air is injected, a balloon near the tip of the catheter is briefly inflated in order to occlude the trachea thereby preventing the injected air from escaping. Once the desired volume has been delivered, the balloon deflates, allowing the gas to be exhaled from the lung around the catheter (between the catheter and the tracheal wall). Doing so cuts the anatomical dead space by the volume of the upper airways and the trachea (˜100 ml in an adult) and further reduces the dead-space dynamically by virtue of the aerodynamic mixing of the injected jet. The tidal volume can thus be reduced by the same amount of dead-space reduction thereby cutting down pulmonary pressure and chest expansion.

We disclose active Exhalation or evacuation of the air from the lung by applying controlled phased and synchronized negative pressure at the air outlet (glottis, mouth or nose) by means of a laryngeal mask or mouth-nose face mask.

### Definitions

The term readily connectable, as referred to herein, should be construed as including any structure and/or member that is configured to be conveniently connected to other structure and/or member and/or components of a larger system or assembly. The term readily connectable, however, doesn't necessarily mean readily disconnectable or removable. The term readily connectable is optionally satisfied by providing for ease of onetime connection or coupling.

By operationally connected and operably coupled or similar terms used herein is meant connected in a specific way (e.g., in a manner allowing fluid to move and/or electric power or signal to be transmitted) that allows the disclosed system and its various components to operate effectively in the manner described herein.

The terms elastic or resilient, as referred to herein, are to be construed as having tensile strength lower than aforesaid tensile strength of pliable or pliant material and optionally being capable of efficiently stretching or expanding, relating inter alia to essentially ductile materials, having UTS value lesser than about 600 MPa.

The terms method and process as used herein are to be construed as including any sequence of steps or constituent actions, regardless a specific timeline for the performance thereof. The particular steps or constituent actions of any given method or process are not necessarily in the order they are presented in the claims, description or flowcharts in the drawings, unless the context clearly dictates otherwise. Any particular step or constituent action included in a given method or process may precede or follow any other particular step or constituent action in such method or process, unless the context clearly dictates otherwise. Any particular step or constituent action and/or a combination thereof in any method or process may be performed iteratively, before or after any other particular step or action in such method or process, unless the context clearly dictates otherwise. Moreover, some steps or constituent actions and/or a combination thereof may be combined, performed together, performed concomitantly and/or simultaneously and/or in parallel, unless the context clearly dictates otherwise. Moreover, some steps or constituent actions and/or a combination thereof in any given method or process may be skipped, omitted, spared and/or opted out, unless the context clearly dictates otherwise.

In the specification or claims herein, any term signifying an action or operation, such as: a verb, whether in base form or any tense, gerund or present/past participle, is not to be construed as necessarily to be actually performed but rather in a constructive manner, namely as to be performed merely optionally or potentially.

The term substantially as used herein is a broad term, and is to be given its ordinary and customary meaning to a person of ordinary skill in the art (and is not to be limited to a special or customized meaning), and refers without limitation to being largely but not necessarily entirely of that quantity or quality which is specified.

The term essentially means that the composition, method or structure may include additional ingredients, stages and or parts, but only if the additional ingredients, the stages and/or the parts do not materially alter the basic and new characteristics of the composition, method or structure claimed.

As used herein, the term essentially changes a specific meaning, meaning an interval of plus or minus ten percent (±10%). For any embodiments disclosed herein, any disclosure of a particular value, in some alternative embodiments, is to be understood as disclosing an interval approximately or about equal to that particular value (i.e., ±10%).

As used herein, the terms about or approximately modify a particular value, by referring to a range equal to the particular value, plus or minus twenty percent (+/−20%). For any of the embodiments, disclosed herein, any disclosure of a particular value, can, in various alternate embodiments, also be understood as a disclosure of a range equal to about that particular value (i.e. +/−20%).

As used herein, the term or is an inclusive or operator, equivalent to the term and/or, unless the context clearly dictates otherwise; whereas the term and as used herein is also the alternative operator equivalent to the term and/or, unless the context clearly dictates otherwise.

It should be understood, however, that neither the briefly synopsized summary nor particular definitions hereinabove are not to limit interpretation of the invention to the specific forms and examples but rather on the contrary are to cover all modifications, equivalents and alternatives falling within the scope of the invention.

### DESCRIPTION OF THE DRAWINGS

The present invention will be understood and appreciated more comprehensively from the following detailed description taken in conjunction with the appended drawings in which:

FIG. 1 is a flow diagram of the main physiological events during cardiac arrest and upon onset of CPR;

FIG. 2 is a block diagram of the unified invention;

FIG. 3 is a schematic overview of an embodiment of the unified invention;

FIG. 4 is a schematic drawing of an ETCO2-based servo-controlled carbon dioxide rich gas supply for ventilation and heart-lung machine;

FIG. 5 is a schematic drawing of a CPR-Synchronized ventilation and active exhalation;

FIG. 6A is a flow diagram of airways-pressure servo-controlled ventilation triggering apparatus;

FIG. 6B is a schematic diagram of airways-pressure servo-controlled ventilation triggering process;

FIG. 7 is an intra-tracheal ventilation Prior art from U.S. Pat. No. 7,513,256;

FIG. 8A is an example of a distal-to-proximal sequentially inflating Exsanguination wrap for CPR;

FIG. 8B is an example of a spring-loaded one-way valve for distal-to-proximal sequential exsanguination wrap for CPR;

FIG. 8C is an example of use of distal-to-proximal sequentially inflating Exsanguination wrap for CPR;

FIG. 9A is an example of a distal-to-proximal sequentially rolling Exsanguination turnstiles for CPR;

FIG. 9B is an example of assembly of distal-to-proximal split-sleeve sequential rolling Exsanguination tourniquets for CPR;

FIG. 9C is an example of near-completion assembly and completed distal-to-proximal split-sleeve sequential rolling Exsanguination tourniquets for CPR;

FIG. 10A is a required fraction of carbon dioxide-FCO2 in delivered gas in order to achieve 4 exemplary levels of arterial PCO2-PaCO2 as function of rate of tissue carbon dioxide product-VdotCO2;

FIG. 10B is a required fraction of carbon dioxide-FCO2 in delivered gas in order to achieve an arterial PCO2-PaCO2 of 55 mm Hg with exemplary 4 tidal volumes-VT as function of rate of tissue CO2 product-VdotCO2;

FIG. 10C is a required fraction of carbon dioxide-FCO2 in delivered gas in order to achieve an arterial PCO2-PaCO2 of 55 mm Hg with exemplary 4 respiratory rates—f, as function of rate of tissue carbon dioxide product-VdotCO2;

FIG. 10B is a required fraction of carbon dioxide-FCO2 in delivered gas in order to achieve an arterial PCO2-PaCO2 of 55 mm Hg with exemplary 4 levels of Dead Space-VD, as function of rate of tissue carbon dioxide product-VdotCO2;

FIG. 11A is a required fraction of O2-FO2 in delivered gas in order to achieve 4 exemplary levels of partial pressure of molecular oxygen in arterial blood-PaO2 as function of rate of tissue molecular oxygen consumption-VdotO2;

FIG. 11B is a required fraction of molecular oxygen-FO2 in delivered gas in order to achieve partial pressure of Oxygen in arterial blood-PaO2 of 161 mm Hg with 4 exemplary levels of Tidal Volume—VT as function of rate of tissue molecular oxygen consumption—VdotO2;

FIG. 11C is a required fraction of molecular oxygen-FO2 in delivered gas in order to achieve partial pressure of Oxygen in arterial blood-PaO2 of 161 mm Hg with 4 exemplary levels of Respiratory Rate-f as function of rate of tissue molecular oxygen consumption-VdotO2;

FIG. 11D is a required fraction of molecular oxygen-FO2 in delivered gas in order to achieve partial pressure of Oxygen in arterial blood-PaO2 of 161 mm Hg with 4 exemplary levels of Dead Space-VD as function of rate of tissue molecular oxygen consumption-VdotO2;

FIG. 12A is an exemplary modus operandi of the Gas Exchange Calculator;

FIG. 12B is a graphic representation of the relationships between the parameters.

While the invention is susceptible to various modifications and alternative forms, specific embodiments thereof have been shown merely by way of example in the drawings. The drawings are not necessarily complete and components are not essentially to scale; emphasis instead being placed upon clearly illustrating the principles underlying the present invention.

### DETAILED DISCLOSURE OF EMBODIMENTS

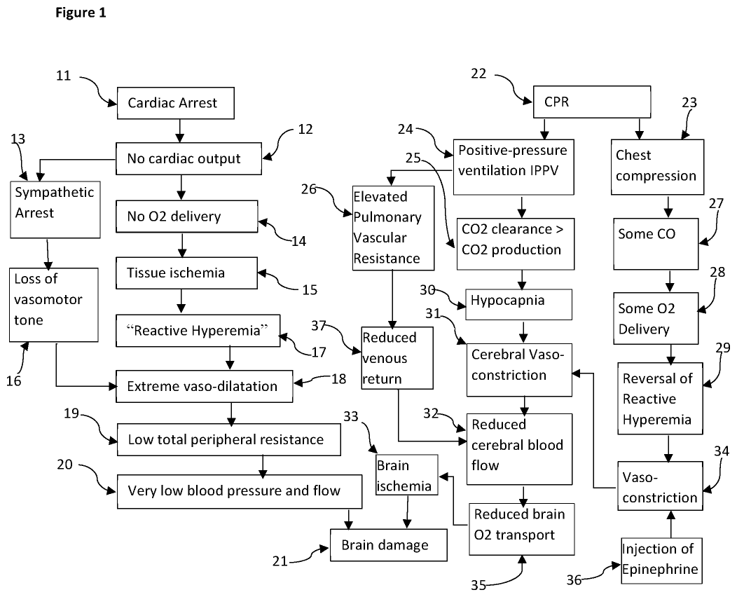

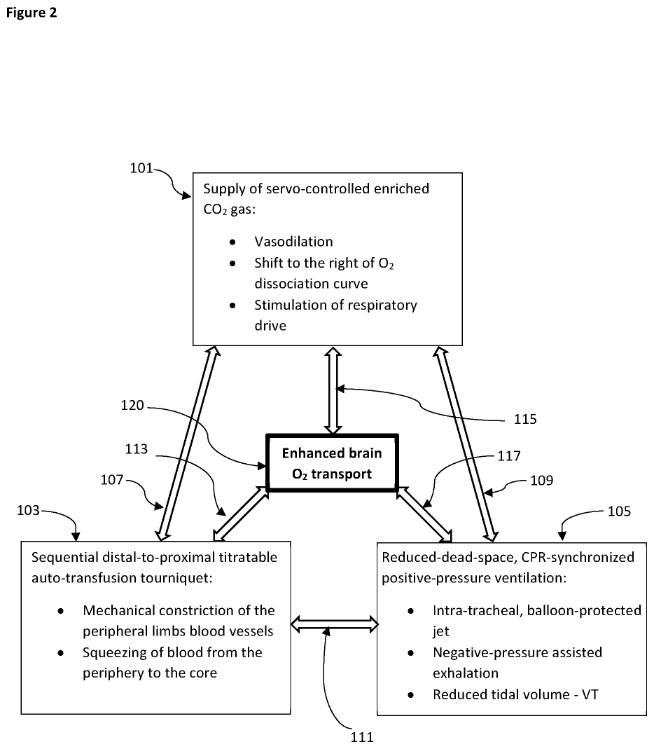

In accordance with the present invention, a triad of means is used to optimally treat a cardiac arrest patient undergoing cardiopulmonary resuscitation (CPR) so that maximal amount of oxygen reaches the brain. FIG. 2 shows a schematic block diagram of the invention which integrally and inseparably consists of 3 elements. As explained herewith, these elements interact clinically and physiologically with each other in a way that not only enhances their collective effect on oxygen supply to the brain, but also, when one of the elements is not present, the effect of the others may be detrimental and cause harm, rather than benefit. This is best understood by reviewing shortcomings of the current art of administering CPR in accordance with the teaching of the American Heart Association and outlined in the block diagram of FIG. 1.

The left side of the diagram of FIG. 1 outlines the physiological events immediately after cardiac arrest occurs. The right side describes the consequences of the current treatment regime that is collectively known as Cardio-Pulmonary Resuscitation—CPR. The result, as described in the introduction, is that in many cardiac arrest patients treated with the current regime, the heart resumes beating (also known as Return of Spontaneous Circulation—ROSC), but suffer devastating and irreparable damage to their brain. The overall outcome is that only a small number of patients undergoing CPR experience meaningful survival with acceptable mental functionality. The sequence of events starts with the acute event of cardiac arrest 11 with the resulting cessation of cardiac output 12. As a result, two major events occur: Oxygen is not delivered to all the tissues 14 and the sympathetic nervous system 13, which controls the tone of the smooth muscles in the arterioles and other blood vessels stop functioning. The lack of blood flow to all of the tissues 15 causes compensatory vasodilatation 18 also known as Reactive Hyperemia 17 this adds up to the effect of sympathetic arrest 13 and the resulting loss of vasomotor tone 16. The extreme vasodilation 18 reduces the total peripheral resistance 19 and causes a fall in blood pressure and flow when cardiac massage starts as part of CPR. The widening of the peripheral blood vessels causes pooling of blood in the periphery so that most of the blood does not return to the heart to fill the heart chambers 37. In summary of the left side of the diagram it can be simply said that cardiac arrest causes extreme vasodilation.

When CPR 22 is administered the patient receives external chest compressions 23 at a rate of 100 compressions per minute, artificial positive pressure ventilation 24 and, according to the current AHA protocol epinephrine is injected 36. When effective chest compression is started some cardiac output 27 and some oxygen deliver 28 are generated, which reverse, to a degree, the mechanisms that cause reactive hyperemia 29 with the ensuing vasoconstriction 34. IV injection of epinephrine 36 also causes vasoconstriction 34, primarily of the brain blood vessels 31 because the brain is first to receive blood from the heart. The onset of positive pressure ventilation IPPV 24, even at 1 or 2 breaths every 15 compressions, i.e. 6 to 12 breaths per minute, clear more CO2 from the lung than the CO2 produced in the tissues 25, which rapidly drops the arterial partial pressure of CO2 known as hypocapnia 30. Hypocapnia 30 has a direct effect on the cerebral circulation by causing vasoconstriction 31 and substantial drop in cerebral blood flow 32 and O2 transport 35. Hypocapnia also cause shift to the left of the Oxygen-hemoglobin dissociation curve which means that for every ml of blood flowing through the tissue, less oxygen is departing from the hemoglobin and handed to the tissue. This contributes to the reduced O2 transport to the brain 35 among other tissues.

Another detrimental effect of Positive Pressure Ventilation IPPV 24 is the ensuing expansion of the lungs and the chest and the elevation of intrathoracic pressure. The elevation of intrathoracic pressure during IPPV further reduces the return of venous blood to the right side of the heart 37 by diminishing the pressure gradient from the veins outside the chest to the vena cava segments inside the chest. At the same time, the expansion of the lung by IPPV results in elevation of pulmonary vascular resistance 26 that impedes the blood flowing from the right side of the heart to the left, thereby reducing venous return to the left ventricle. The fact that the filling of both heart chambers is reduced by IPPV is well-known and adds up to the diminished venous return due to the pooling of blood in the periphery so that the cardiac output achieved by CPR chest compression is less than ⅓rd of normal. The final result is further reduction of blood flow and O2 transport to the brain 35, exacerbation of brain ischemia and, within a few minutes, to permanent brain damage.

Based on the information described above, it is clear that the combination of positive pressure ventilation and epinephrine in the presence of very low cardiac output lead to critically low O2 transport to the brain tissue. As such, this invention teaches that CPR must use the exact opposite approach to protect the brain from being damaged: we must induce and maintain vasodilation, focused to the brain circulation, we should compress and constrict the peripheral blood vessels and we should absolutely avoid impediment of blood flow to the heart caused by IPPV, while shifting the O2-hemoglobine dissociation curve to the right, not to the left. This patent teaches how to do so by combining the processes outline below.

A schematic block diagram of the unified invention is shown in FIG. 2. The most potent and natural means to vasodilate the arterioles and other blood vessels is by inducing hypercapnia which is elevated partial pressure of Carbon Dioxide CO2. This is easily done by supplying CO2-enriched gas 101 to the ventilator circuit and, if used, into the gas-exchanger in a heart-lung machine such as ECMO or bubble oxygenator. As listed briefly in 101 enriched CO2 induces vasodilatation, shifts the O2-Hemoglobin dissociation curve to the right to facilitate transferring to O2 to the cells and their mitochondria, and stimulate the patient's respiratory drive. However, CO2-induced vasodilation is global, leading to pooling of blood in the periphery and as such, by itself, is not at all helpful. Means to counteract 107 the peripheral vasodilatation must be included. This invention teaches combining the use of means to squeeze blood from the periphery into the core by applying a Sequential distal-to-proximal auto-transfusion and preventing return of the blood into the periphery by acting as a tourniquet 103. The combined effect of 103 is to shift blood from the periphery to the core thereby increasing the Preload to the heart, while restricting the flow to the periphery thereby channeling the CPR-generated flow to the essential organs and minimizing the shunting of blood which results in higher resistance to flow also known as the Afterload of the heart.

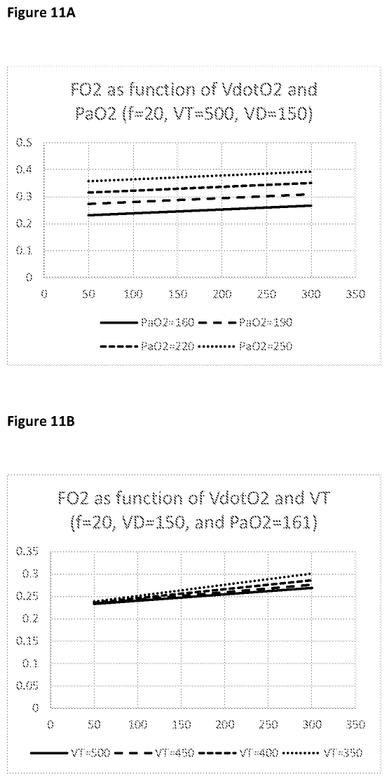

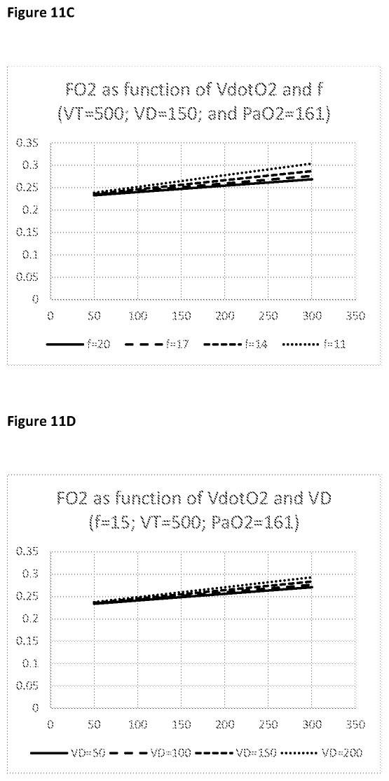

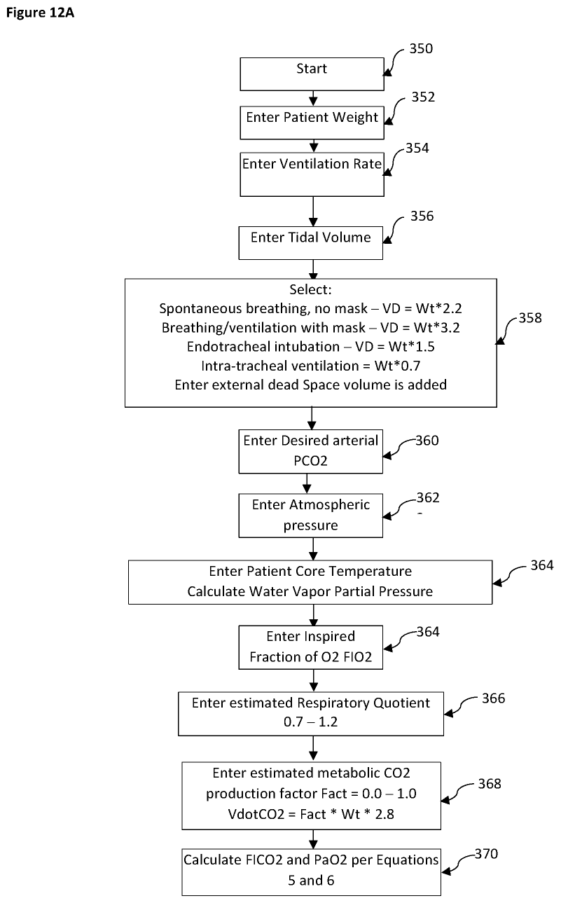

The increase of venous return to the right heart by sequentially squeezing the limbs as taught in 103 does not completely overcome the impediment to blood flow imposed by IPPV 111. This means that artificial ventilation must be modified 105 in order to minimize the increase in pressure and distention during ventilation. To do so this invention teaches minimizing the tidal volume by reducing the anatomical dead-space by injecting the inspiratory fresh gas into the distal trachea and doing so in synch with the decompression (passive or active) of the CPR. The component of the invention of 105 also discloses applying negative pressure (suction) during the expiratory phase of the ventilation cycle. It also teaches reducing the tidal volume—VT to a level that is sufficient to bring arterial O2 saturation to 98% and not more. This is done according to a mathematical algorithm described in FIGS. 11a, 11b, 11c, and 11d that takes into account all the factors that influence O2 transport including tidal volume, dead space, respiratory rate, arterial PCO2, P50 (the position of the O2—dissociation curve), the rate of O2 consumption by the tissues and the use (if any) and efficacy of a heart-lung machine.

The ventilation scheme described in 105 tightly interacts 109 with the supply of CO2 described in 101 through servo control of the delivered CO2 fraction FCO2 in the inspired gas or in the heart-lung machine exchanger. For any desired arterial partial pressure of CO2 for 101, FCO2 is influenced by the parameters of the ventilation of 105 and vice versa.

The combined effect of all 3 poles of this invention effectively influences the cerebral blood flow extent of O2 transport to the brain tissues 120 as shown schematically by the processes indicated by reciprocal arrows 113, 115 and 117.

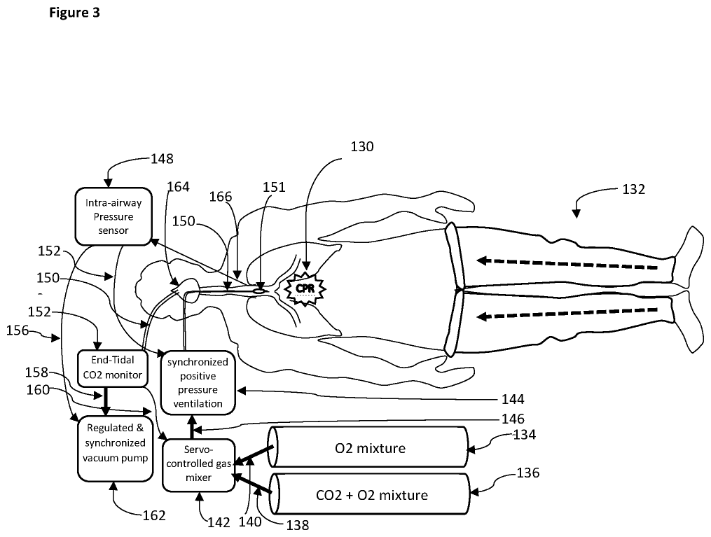

In order to better understand the interrelated poles of this invention, we now refer to FIG. 3 where CPR chest compression 130 of a cardiac arrest victim is shown. First, we point to the sequentially applied distal-to-proximal limb compression and tourniquet device 132. This is the means by which the vasodilating effect of the CO2-rich gas is counteracted, blood is shifted from the limbs to the core and prevented from returning. One can realize without showing so in the drawing that such limb compression device can be applied also on the arms of the victim. Next, is shown the gas mixer 142 that is servo-controlled by receiving continuous information via wired or wireless transmission 160 about the level of end-tidal CO2 in the exhaled air. The end-tidal CO2 is tracked by a CO2 analyzer 152 which is in direct communication 150 with a face or laryngeal mask 164 where said exhaled gas is transmitted through. The level of CO2 partial pressure at the end of exhalation corresponds to the arterial partial pressure of CO2 and therefore can be used in order to servo-control the gas mixture by the mixer 142 fed into the ventilator 144 via conduit 146. Other embodiments of this part of the invention is by using trans-cutaneous CO2 monitor or indwelling arterial CO2 electrode not shown in this drawing. Yet another embodiment of the invention is by splitting the gas mixture passing through conduit 146 and feeding a portion of it into the gas-exchanger of a heart-lung machine 163 also not shown in FIG. 3. The Servo-controlled gas mixer 142 mixes gases from a plurality of compressed gas cylinders 134 and 136 connected to the mixer 142 via corresponding conduits 138 and 140. In a preferred embodiment, one cylinder contains compressed pure (100%) oxygen and the other contains a mixture of 5% CO2 also known as Carbogen. Mixing the two gases in any proportion, can generate FCO2 levels between 0.0 (if all the gas comes from the 100% O2) to 0.05 (if all the gas is supplied by the Carbogen cylinder). Other embodiments consist of higher CO2 concentration in the Carbogen cylinder including levels between 5- and 6% or 6- and 8%. An additional embodiment consists of adding inert gases such as Xenon or Argon to the O2 mixture in proportions of 35% to 45% O2 with corresponding levels of 65% to 55% of the inert gas. The same proportion of Oxygen needs to be included with the inert gas-Carbogen mixture that is to say lower level of inert gas to verify that the patient always receives enough O2.

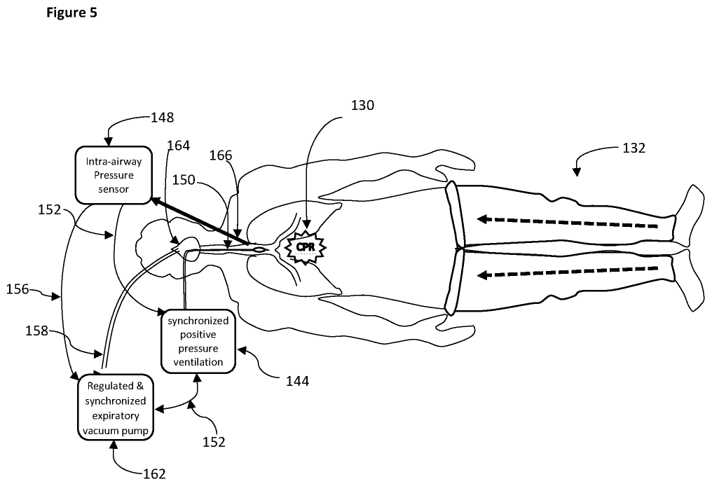

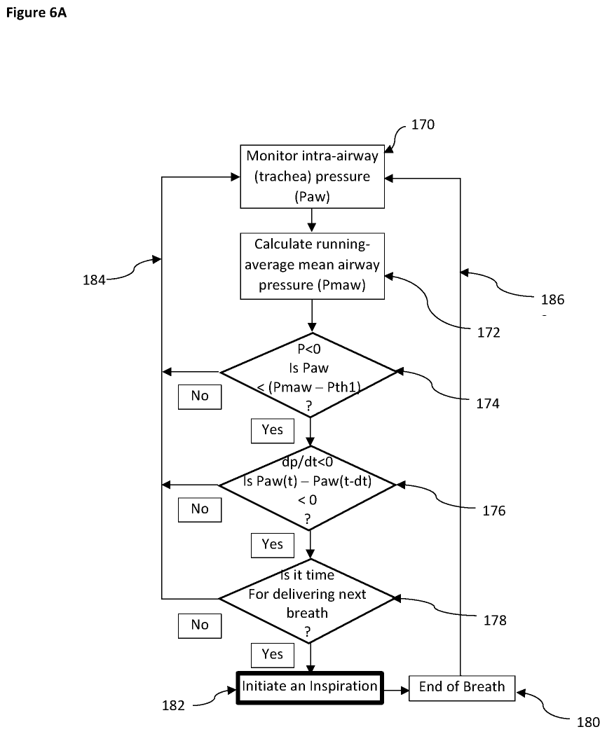

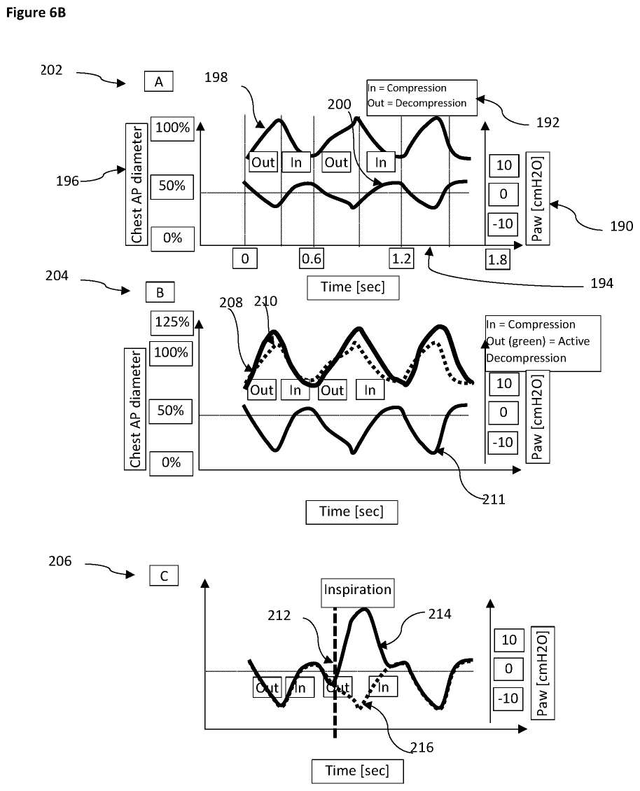

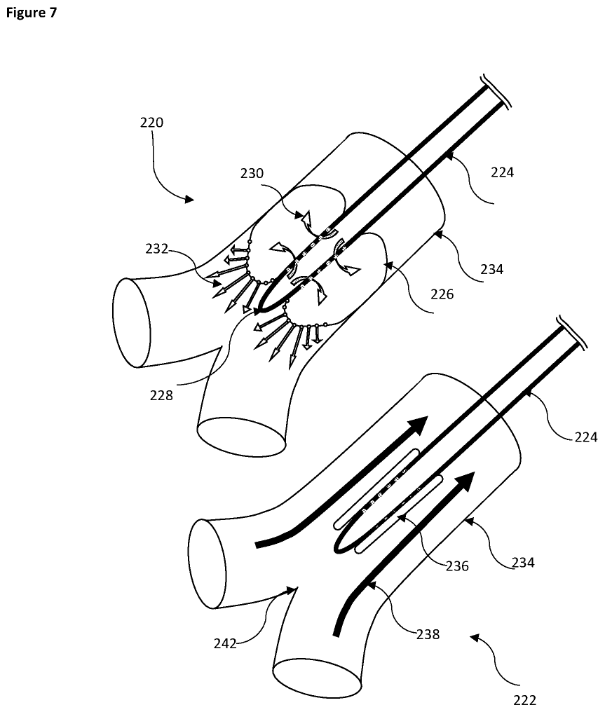

Next, we disclose in FIG. 3 the elements that are used to ventilate the patient in a way that does not impede blood flow into the chest and through the lungs. These are regulated and servo-controlled by a high-fidelity pressure sensor 148 in communication 166 with the patient's airways. The signal is analyzed by a computer-based algorithm shown in FIG. 6 a that determines if it is time to initiate a breath by the synchronized positive pressure ventilator 144 or to initiate vacuum evacuation of the gas by pump 162. In a preferred embodiment, the positive pressure ventilator 144 injects the gas into the distal portion of the trachea via a narrow catheter 150 of an internal diameter of 4+/−1 mm. As soon as gas starts flowing into the catheter 150, an elastic balloon 151 inflates to occlude the trachea and prevent escape of the air. This intra-tracheal ventilation is described in detail in FIG. 7 and in U.S. Pat. No. 7,513,256. Injecting the gas at the distal trachea and having it exit around the catheter 150 verify that the fresh gas interface moves from the airway opening (mouth/nose) to a deeper position, thereby cutting the dead space by approximately 100 cc in an adult. Doing so allows cutting the tidal volume by the same amount so that the lung and chest expansion are reduced. Another element of this preferred embodiment is the active evacuation of the gas via the mask 164 and the conduit 150 aided by the regulated and synchronized vacuum pump 162, which is servo-controlled 156 by the signal from the intra-airway pressure sensor 148.

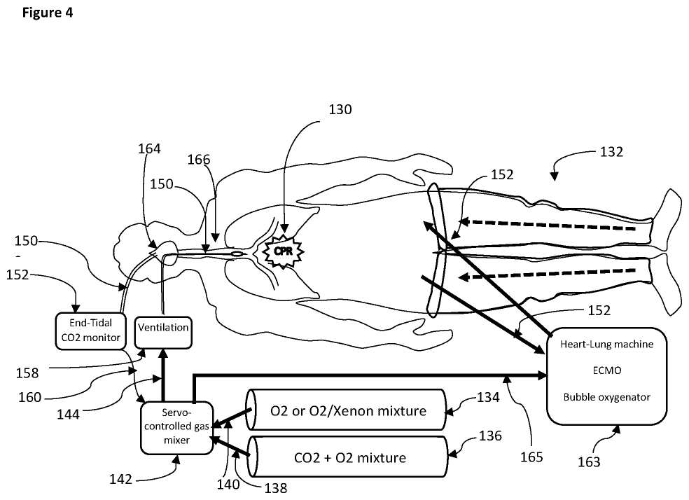

It is now possible to further describe the end-tidal CO2-based servo control of the composition of the gas supply for the ventilator and the heart-lung machine as shown in FIG. 4. In this Figure we reduce the clutter by focusing on the gas delivery. The ventilator 158 and the heart-lung exchanger 163 receive gas from the servo-controlled gas mixture 142 that mixes a plurality of gases 134 and 136 supplied from compressed gas cylinders via conduits 138 and 140. The gas mixture receives a signal from the End-tidal CO2 monitor 152 and adjust the level of CO2 in the mixture to verify that the Partial pressure ETCO2 is at the desired level. It is clear to an expert in the field that if the CO2 level in the exhaled gas is higher than the desired level, the FCO2 in the gas must be reduced and if it is lower than the desired, the FCO2 in the gas mixture should be increased. The feedback control is such that changes are made fast enough to be effective, yet not to fast in order to avoid undesired undulations of CO2 (under-damped feedback loop).

Description of another portion of the invention is shown in FIG. 5 where the pumping of air into the lung and the evacuation of air out of the lung are synchronized with the intra-airway fluctuations caused by the CPR chest compressions. The preferred embodiment is based on using a pressure signal obtained with an intra-airway pressure sensor 148, which is either inserted into the airways or connected to the airway lumen via a conduit 166. The pressure sensor should be sensitive with sufficient frequency response from 0 Hz to 100, or 0 Hz to 1000 Hz to the changes in pressure inside the airways induced by the CPR chest compressions. The pressure sensor can be free-standing or incorporated into the air-delivery catheter 150. Another preferred embodiment uses the signal from a chest motion accelerometer which tracks the vertical motion of the chest wall. In yet another embodiment, the pressure signal is obtained from an esophageal pressure sensor placed in the lower ⅓rd of the esophagus. Another preferred embodiment uses the central venous pressure (CVP) to monitor and track the changes in intrathoracic pressures due to CPR chest compressions. It is also clear that any combination of such sensors can be used in order to obtain a more accurate and robust data for triggering the onset of inspiration delivered by the positive pressure ventilator 144 and the active expiration air evacuation vacuum pump 162. The trigger signal is used in the preferred embodiment to initiate the delivery of gas according to the flow diagram algorithm shown in FIG. 6 a and schematically in FIG. 6b. The pressure signal is also used to initiate and control the evacuation of the air from the lung by verifying that the airway pressure is mildly sub-atmospheric but not too low and kept in the range of −2 to −7 cm H2O. The control of the vacuum is by opening and closing a solenoid valve or a MEMS component or by controlling the speed of a motorized or an electromagnetic vacuum pump. The control and the action of the evacuation pump is not shown in the drawings. The preferred embodiment of the gas delivery positive pressure ventilation 144 is shown in FIG. 7 as the intra-tracheal ventilator previously patented in the U.S. Pat. No. 7,513,256. Other types of volume or pressure ventilators that can be externally triggered can also be used.

The preferred embodiment of the algorithm used for transforming the pressure signal into air delivery activation trigger is shown in FIG. 6a. A signal corresponding to the instantaneous chest compression is obtained 170 as an intra-airway pressure signal. As disclosed earlier, other signal such as chest wall acceleration, proximity signal, optical reflection signal, auxiliary signal from a mechanical compression device, tracheal tube signal or intra-vascular pressure signal can also be used. The running average mean value of the chest pressure or position is calculated over time 172 to determine the reference point. This is done by using a digital method or an analog integrator. As long as the chest diameter or pressure signal shows that it is above the said average value Pmaw determined by using the “if” box 174, the path of the algorithm is looped through 184 to continue acquiring the signal via 170. This loop is repeated until a value of chest diameter or pressure indicates that the chest diameter is below Pmaw. Once this is detected, a second test is applied to verify that the chest diameter or pressure is trending towards further decreasing diameter or pressure by 176 by calculating the first derivative of the diameter or pressure signals. If the gradient is not towards continued decrease of diameter or pressure, the looping via path 184 to 170 continues. Once both actual size and the size derivative are indicating the onset of a decompression phase of the chest, the algorithm then checks if it is the right time to initiate a breath 178. If not, looping through path 184 to 170 continues at a sufficient rate, for example every 5 msec. Once the test in 178 detects that it is time to initiate a breath, either by determining the elapsed time since the previous breath, which can be, for example, 5 or 10 or 12 seconds or by determining the number of chest compressions performed since the previous breath, which can be, for example, 15 compressions or 7 or 8 compressions, the algorithm initiates an inspiration 182. This is done by activating positive air flow into the patient's trachea via the inserted catheter or tube, an example of which is shown in FIG. 7. A timer or a volume counter or a pressure signal from the airways is then activated and when a threshold indicating that enough gas has been injected into the lungs the End of Breath decision 180 is activated and the injection of air is stopped and control path 186 returns the algorithm to repeat the process at 170.

A schematic implementation example of the breath initiation process is shown in FIG. 6b which consists, for the sake of clarity, of 3 panels. Panel A 202 shows the chest anterior-posterior (AP) diameter of the chest 196 as a continuous tracing 198. When the tracing is upward (“OUT”) 192 it means that the chest diameter is getting bigger. In this panel A the decompression is by passive elastic recoil of the chest wall. The time axis 194 shows in seconds the elapsed time starting from an arbitrary time-zero. The second tracing in panel A, marked as 200, shows the airway pressure (Paw) 190 in cm H2O where zero is referenced to atmospheric pressure. It is possible to see that when the chest is compressed (in), the pressure increases and when it is decompressed (out) Paw falls and may even become negative relative to atmosphere.

Panel B similarly shows the chest motion in the upper tracing as the passive recoil 210 as in panel A and also as when an active suction or decompression mechanism that pulls the chest outward is activated 208. Both tracing 208 and 210 refer to the chest diameter axis on the left side of the panel. Active decompression can actually expand the chest to have a higher AP diameter and volume than with the passive recoil. Moreover, as shown in the pressure tracing 211 this can bring the intra-airway pressure below atmospheric level as referenced to the second y axis on the right. Timing the delivery of gas into the lung to coincide with this negative decompression phase clearly shows the advantage of this invention over the existing art which does not synchronize the delivery of the gas with the chest compressions, resulting in higher intra-thoracic and intra-airway pressure. As explained before, this has the advantage of minimizing the impediment to blood flow from the main veins into the right side of the heart. This is shown schematically in panel C where the algorithm detects that (a) the pressure is negative; and (b) it is monotonically declining (dp/dt is positive), so that the initiation of a breath is triggered 212. The balance of the negative pressures of the decompressed chest 216 and the positive pressure of the delivered gas is giving a pressure tracing 214 that is lesser than if no synchronization was accomplished.

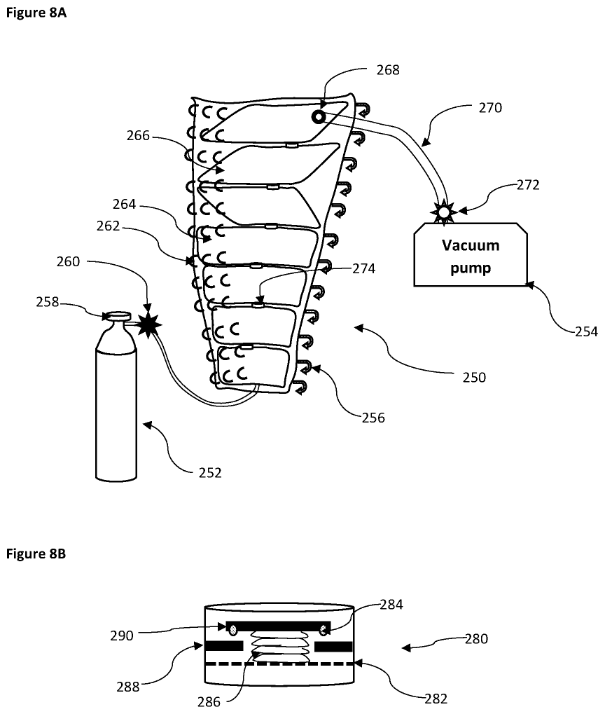

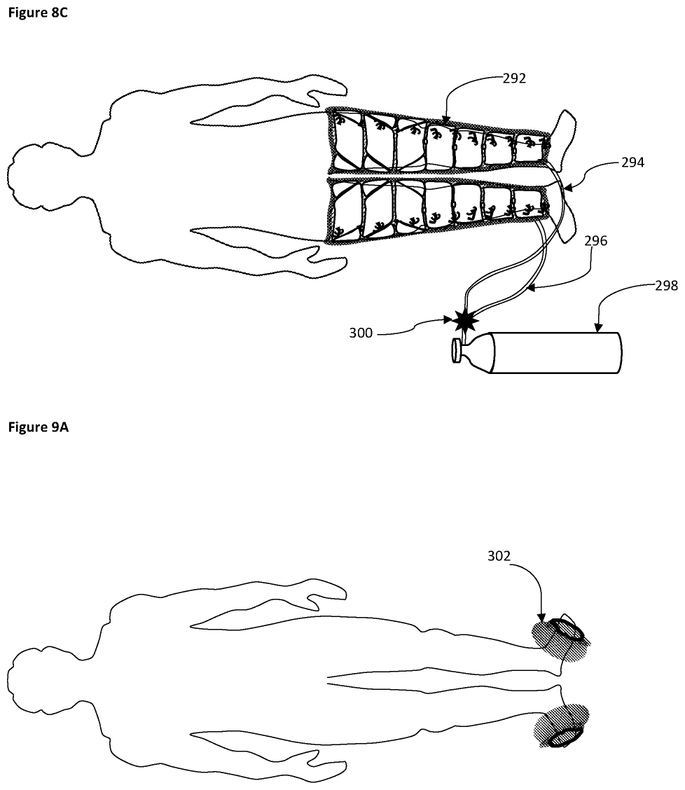

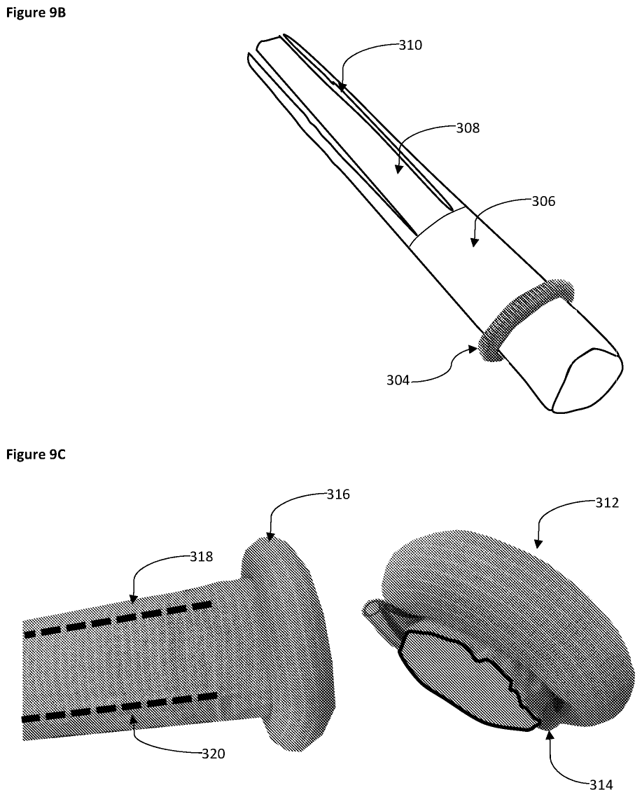

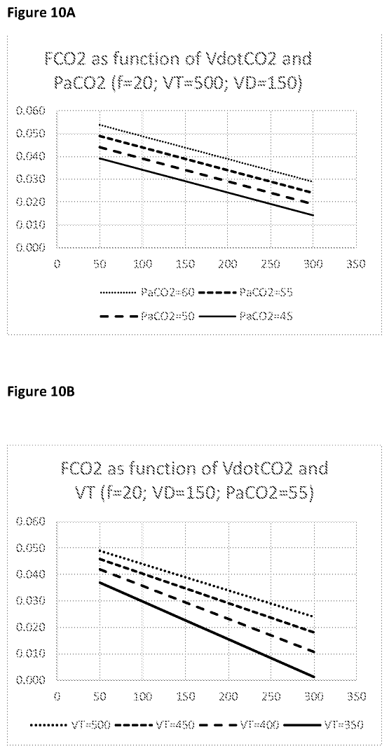

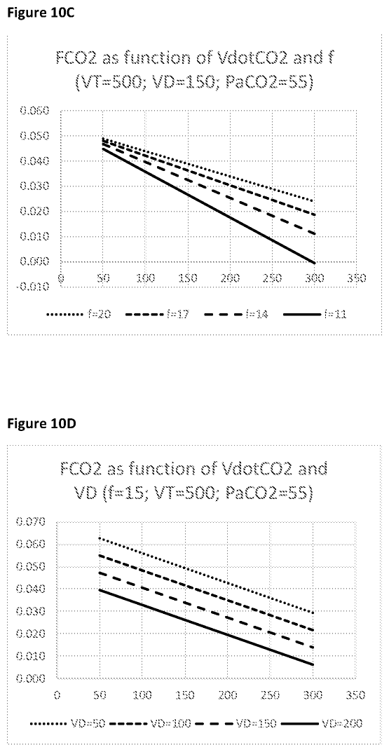

FIG. 7 incorporates the teaching of U.S. Pat. No. 7,513,256 as an optimal and preferred embodiment for combining with the other two modalities of this patent. Although other modes of positive pressure ventilation can be used and the triggered initiation of a breath may be accomplished, they may not have quick enough response time to be accurately synchronized with a 100 compressions per minute CPR. The intra-tracheal ventilator operates with a narrow catheter, which is typically half of that used with normal endotracheal tubes. Halving the diameter requires increasing the driving pressure 16 fold in the ventilator, which reduces the volume of air in the ventilator (according to Boyle's law) also 16 fold. This means that the size of the ventilator can be reduced by the same proportion which makes it frequency response much quicker and much more suitable for ventilation in synch with CPR. In addition, the fact that the outflow of air is around the narrow tube, means that the cross-sectional area available for evacuation of the lung is much higher thereby dramatically decreasing the resistance to evacuation of the lung during the expiratory phase of the ventilation cycle. This helps further reduce the average lung and chest volume and reduce the impediment to blood return to the right heart. Adding the synchronized active evacuation of the gas by incorporating a vacuum pump further reduces the lung and chest volume. It should be noted that the practice of sucking on the veins by creating a negative intra-thoracic pressure, whether by active chest decompression, by using an Inspiratory Threshold Device or by applying vacuum during exhalation is substantially flow-limited by the collapsible nature of the large veins. It is therefore necessary to apply the third component of this invention, namely the sequential distal-to-proximal limb compression shown in FIGS. 8 and 9, which pushes the blood from the periphery to the core rather than attempting to syphon it.

Shifting blood from the limbs to the core during emergencies is an old practice. Lifting the leg is described in old texts and, in fact, is used in orthopedic surgery a means of exsanguination prior to inflating the pneumatic tourniquet in order to create a bloodless surgical field. According to studies by Blond et Al published in Acta Orthopedica Scandinavia in 2001-2, about 45% of the blood is shifted from the limbs by limb elevation. This means that 55% of the blood remains in the limbs. Attempts to use the Medical Anti Shock Trousers for this purpose did not work well (Bickel et al. Ann Emerg Med. 1987 June; 16 (6): 653-8.) a device called HemaClear (RTM) www.hemaclear.com is widely used to shift blood from the limbs to the core and block its reentry in orthopedic surgery and a similar device called HemaShock (RTM) www.hemashock.com is available for emergency use. The current invention discloses two additional devices that are uniquely suitable for quickly and effectively squeezing the blood from distal-to-proximal during cardiac arrest as part of this CPR mode to counteract the vasodilating effect of using Carbogen and for priming the heart while increasing the afterload, the diastolic blood pressure, the coronary perfusion pressure and, most importantly, the cerebral blood flow.

In FIG. 8a we disclose a pneumatic distal-to-proximal sequentially inflating Exsanguination wrap for CPR 250 for applying on a single limb. The device consists of multiple inflatable bladders 264 embedded in a fabric envelope 262. The bladders are rectangular or paralleloid-shape 266 to facilitate optimal cover and compression of the limb when inflated. The wrap is applied quickly on the limb and closed with hook-and-loop fasteners 256 or similar secure and adjustable closures. Inflation starts from the most distal compartment and is preferably done by a connecting a compressed-gas cylinder controlled by a flow regulator 258 and a pressure regulator 260. Once the pressure in the most distal bladder reaches a pre-set level controlled by a spring-loaded one way valve 274 described in FIG. 8b, the second bladder starts to be filled up until all the compartments are inflated to the desired pressure. As such, the wrap compresses the limb sequentially from distal to proximal. Inflation can be done in an alternative embodiment by using manual or motorized pump (not shown). The process of deflation of the wrap must be stepwise and from proximal to distal. The bladders should be deflated one at a time and after each bladder is deflated the patient's vital signs must be evaluated. The evacuation is done by applying vacuum through a vacuum pump 254 through a regulator 272 and a thick-wall tube 270 connected to a valve 268. Note that after the most proximal bladder is emptied, the spring-loaded one-way valve in between the bladders will only open when the vacuum level becomes higher than the threshold of opening the spring-loaded valve. As such, the bladders will deflate one at a time as required.

A preferred embodiment of the pressure-regulating one-way valves between the bladders is shown in the schematic drawing 280 of FIG. 8b. The valve plate 290 is pulled by the spring 286, firmly attached on its bottom side to the fenestrated stiff support 282. When closed shut, the plate 286 rests snugly on the circular shelf 288 with the elastic cushioned O-ring 284 shown cut acting as a seal between the spring-loaded plate 290 and the occluding shelf 288. There is a spring-loaded one-way valve between all the bladders as shown in FIG. 8a. In order to be opened, the pressure difference between the bottom and the top (distal and proximal) ends of the valve must be sufficient to overcome the pulling force of the spring and in a preferred embodiment should be greater than 200 mm Hg. In other embodiments the force of the spring is set to require pressure difference of 100-150 mm Hg or 150-200 mm Hg or 200-300 mm Hg. The pressure difference can be created either by inflating the distal bladder to above the opening threshold or by applying vacuum that is higher than the valve-opening threshold. We further disclose the ability to apply a wrap 292 to each leg as shown in FIG. 8c. This is done by connecting an inflating tube to each or the wraps such as shown in 294 and 296 that connect to a compressed gas cylinder 298 via the pressure regulator 300. It is clear that wraps of similar design but smaller size can be applied to the arms if it is needed. The blood content of each arm in a healthy person is 150 ml while in each leg it is over 500 ml, but the volume may become higher when blood is pooled in the peripheral blood vessels during cardiac arrest.

We now describe a novel configuration of the elastic exsanguination tourniquet as shown in FIG. 9. The device is similar to the previously patented HemaShock in its general configuration consisting of an elastic ring with an elastic sleeve wrapped around it, whereby the ring rolls up the limb when straps that are also wrapped around the ring are pulled. The unique configuration of the new device is that it does not contain straps. The device 302 is rolled from distal to proximal by pulling the straps 310 or split sleeve 308 shown in FIG. 9b. The preferred embodiment of this exsanguination tourniquet device for CPR consists of a full sleeve initially 306 wrapped around an elastic ring made from an elastic stretchable Silicone or from a metal (steel) spring 304. The rolling of the steel spring ring and the wrapped sleeve 316 up the limb is done by pulling on the straps 310. Once the patient is ready for the exsanguination tourniquet to be removed, it is rolled by hand stepwise from proximal to distal while monitoring vital signs in order to avoid cardio-vascular collapse of the patient.

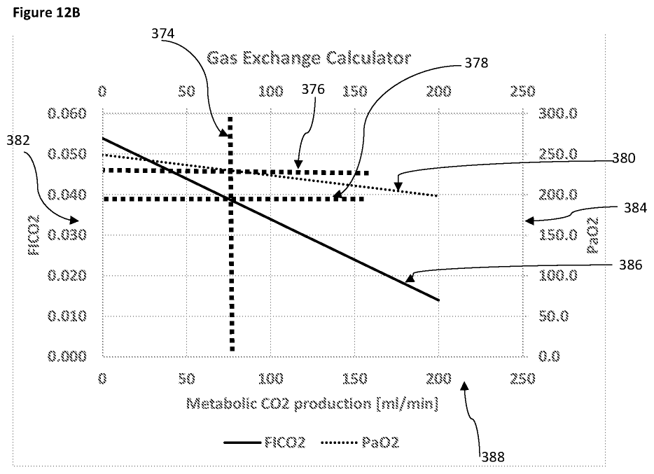

We now turn to the important safety feature of the Gas Exchange Calculator governed by Equations 5 and 6 and uses parameters input calculator of FIG. 12a as shown in the output of FIG. 12b. While the continuous adjustment of the servo-controlled gas mixer 142 is based on feedback from the End-Tidal CO2 Monitor 152, it is safer and more practical to adjust its initial setting and boundaries based on physiological parameters. The Gas Exchange Calculator shown in FIG. 12a determines these initial settings by entering the patient's own parameters. When started at 350 in FIG. 12a, the user first enters the patient's weight 352 followed by entering the ventilation rate 354 and the volume of each breath known as the Tidal Volume 356. Next, the user enters information on the method of ventilation from which the calculator determines the size of the Dead Space VD 358. For example, the VD of a normal person is approximately 2.2 ml/kg when breathing spontaneously. However when a mask is used to ventilate the person, the air in the mask gets re-breathed and is therefore added to the regular anatomical VD to become approximately 3.2 ml/kg. When the patient is intubated with an endotracheal tube, the upper airways are bypassed and the VD is dropped to 1.5 ml/kg. Finally, if the Intra-Tracheal ventilation is used the VD is reduced to 0.7 ml/kg. The user then enters the target PaCO2 360. The influence of the ambient atmospheric pressure and water vapor partial pressure are entered at 362 and 364 where the actual vapor pressure is determined according to the patient's body temperature. The inspired fraction of Oxygen FIO2 is then set by the user 366. An unknown parameter during CPR is how much CO2 the patient is actually generating by the methabolis 368. In a healthy resting individual, it is 2.8 ml/kg/min. The estimate of the actual value during CPR is determined based on the cardiac output generated by the CPR chest compressions. If the cardiac output is 0.3 of the normal cardiac output, the estimated methabolic level should be approximately 0.3.

Using these parameters the Gas Exchange Calculator determines the needed level of FICO2 by Equation 5 and the expected PaO2 by Equation 6. Equation 5 calculates the required FICO2 needed to keep PaCO2 at the desired level as shown in FIG. 12b by line 386. One can see from the graph that the lower the metabolic production of CO2 as shown by the horizontal axis 388, the higher FICO2, indicated by the left side vertical axis 382, should be. If the metabolism is completely shut down and CO2 production is down to zero, the FICO2 must be higher than 0.05. By way of an example, if the metabolic rate is about ⅓ of normal as shown by the vertical dashed line 374, the required FICO2 needed to keep PaCO2 at a level of 55 mm Hg, used as target in this example, should be approximately 0.04 (4%) as shown by the horizonal dashed line 378.

We now turn our focus to the needed level of Oxygen in the inspired gas. Equation 6 calculates the predicted PaO2 from the parameters entered into the Gas Exchange Calculator. Equation 3 dictates the minimum PaO2 needed in order to bring the O2 Saturation to at least 98%. This value is higher than usual because the O2-Hemoglobine dissociation is shifted to the right because of Bohr's effect of the high PaCO2 with P50 at levels as high as 40 mm Hg instead of the normal 26.6 mm Hg. Using Equation 3 we can see that a lower limit of PaO2 of 161 mm Hg is needed. Turning now to Equation 6 and the graph on FIG. 12b we can see line 380 predicting the PaO2. We can also see that at the working point shown by the vertical dashed line 374, the value of line 380 seen as it intercepts with the horizontal dashed line 376 is about 240 mm Hg. This value is higher than the said minimum safe limit of 161 mm Hg. The Gas Exchange Calculator will allow use of these parameters. However, if a combination of parameters is chosen that will give a value of PaO2 at the working point that is lower than 161, the Calculator will generate an alarm signal. As such, the Gas Exchange Calculator is an essential safety feature required when gas mixtures with high values of FICO2 are used.

$$FI {CO}_{2} = \frac{Pa {CO}_{2} \cdot ( VT - V D )}{( P a t m - P {H}_{2} O ) \cdot VT} - \frac{\dot{V} {CO}_{2}}{f \cdot VT} \qquad Equation 5$$

$${Pa O}_{2} = ( P a t m - P {H}_{2} O ) \cdot ( {FI O}_{2} \frac{\dot{V} {CO}_{2} / RQ}{f \cdot ( VT - V D )} ) \qquad Equation 6$$

| Parameter | Units | Value | Name of parameter |
|---|---|---|---|
| f | breaths/min | 10 | Respiratory Rate |
| VT | ml | 500 | Tidal Volume |
| VD | ml | 150 | Dead Space Volume |
| PaCO2 | mm Hg | 55 | Desired arterial PCO2 |
| Patm | mm Hg | 760 | Atmospheric pressure |
| PH2O | mm Hg | 47 | Water vapor pressure |
| FIO2 | pure number | 0.35 | Inspired Fraction of O2 |
| RQ | pure number | 0.8 | Respiratory Quotient |

1-27. (canceled)

28. A system for delivering a synchronized cardiopulmonary resuscitation (CPR), configured to ensure delivery of an efficient amount of oxygen to essential organs whilst prevent cerebral hypocapnia by maximizing delivery of carbon dioxide to a brain, comprises:
(a) at least one limb compression device, configured for exerting a distal-to-proximal sequential compression force onto a limb and for occluding a blood flow into said limb, wherein said compression force is exerted constantly and not intermittently;
(b) a positive-pressure ventilation sub-system, configured for delivering a mixture of gases by positive pressure, comprising:
(I) a carbon dioxide reservoir containing a carbon dioxide enriched gas;
(II) a molecular oxygen reservoir containing a molecular oxygen enriched gas;
(III) a controllable mixing module, operationally connected to said carbon dioxide reservoir and said molecular oxygen reservoir, configured to controllably mix said molecular oxygen enriched gas with said carbon dioxide enriched gas;
(IV) at least one carbon dioxide partial pressure sensor selected from the group consisting of: an arterial blood carbon dioxide partial pressure sensor and end-tidal exhaled air carbon dioxide partial pressure sensor, configured to detect a partial pressure of carbon dioxide in an arterial blood;
(V) a controller, operationally connected to said controllable mixing module and said at least one carbon dioxide partial pressure sensor, configured for controlling at least one ratio selected from the group consisting of: a ratio of said molecular oxygen enriched gas and ratio of said carbon dioxide enriched gas, in a mixture of said molecular oxygen enriched gas and said carbon dioxide enriched gas; wherein said controller is configured to include an efficient amount of said molecular oxygen in said ratio of said mixture of said molecular oxygen enriched gas and said carbon dioxide enriched gas, and
wherein said controller is further configured to increase an amount of said carbon dioxide enriched gas in said ratio of said mixture of said molecular oxygen enriched gas and said carbon dioxide enriched gas, thereby maximizing delivery of carbon dioxide to a brain and preventing cerebral hypocapnia;
(VI) an endotracheal tube comprising a unidirectionally sealing cuff disposed at a distal portion of said endotracheal tube, configured for iteratively and intermittently assuming:
(i) a deployed configuration, wherein said unidirectionally sealing cuff is engaged to an interior surface of a trachea, thereby effectively sealing a passage of gasses in-between said unidirectionally sealing cuff and said interior surface of a trachea, whilst sustaining an inflow of gases from said endotracheal tube, into said trachea;
wherein said deployed configuration is assumed by said unidirectionally sealing cuff during an inhaling phase of a respiratory cycle;
(ii) a withheld configuration, wherein said sealing cuff is disengaged from said interior surface of said trachea, whilst sustaining a spontaneous outflow of said gases from said trachea;
wherein said withheld configuration is assumed by said unidirectionally sealing cuff during an exhaling phase of said respiratory cycle;
(c) a cardiac stimulation device, configured for returning a spontaneous circulation of said arterial blood, by providing at least one type of stimulation to a cardiac muscle, selected from the group consisting of: a mechanical stimulation and electrical stimulation, at a predetermined time intervals;
(d) an intra-tracheal pressure sensor configured for continuously determining a pressure inside said trachea;
(e) a synchronizer configured for timing an injection phase of said positive-pressure ventilation system with an onset of a decompression phase of said cardiac stimulation.

29. The system as in claim 28, wherein said distal-to-proximal sequential compression force onto said limb is achieve by an up-rolling constricting elastic ring.

30. The system as in claim 28, wherein said distal-to-proximal sequential compression force onto said limb is achieved by applying at least one element selected from the group consisting of: an elastic bandage, an elastic limb wrap with adjustable closures, an inflatable limb wrap with adjustable closures.

31. The system as in claim 28, wherein said at least one limb compression device is configured for occluding the arterial inflow of blood into said limb by applying a surface skin pressure range selected from the group consisting of: 100 to 200 mm Hg and 200 to 300 mm Hg.

32. The system as in claim 28, wherein said mixture of gases is selected from the group consisting of: 95% molecular oxygen and 5% carbon dioxide, 0.1 to 2.0% carbon dioxide with the balance being molecular oxygen, 2.1 to 4.0% carbon dioxide with the balance being molecular oxygen, 4.1 to 5.6% carbon dioxide with the balance being molecular oxygen, 0.1 to 5.0% carbon dioxide with 30 to 50% molecular oxygen and the balance being a chemical element Xenon, 0.1 to 5.0% carbon dioxide with 30 to 50% molecular oxygen and the balance being chemical element Argon.

33. The system as in claim 28, wherein said controllable mixing module controllably mixes carbogen gas of 5% carbon dioxide and 95% molecular oxygen with pure 100% molecular oxygen according to feedback from said at least one carbon dioxide partial pressure sensor selected from the group consisting of: said arterial blood carbon dioxide partial pressure sensor and said end-tidal exhaled air carbon dioxide partial pressure sensor, to maintain said arterial blood carbon dioxide partial pressure level at 41 to 45 mm Hg.

34. The system as in claim 28, wherein said controllable mixing module controllably mixes carbogen gas of 5% carbon dioxide and 95% molecular oxygen with pure 100% molecular oxygen according to feedback from said at least one carbon dioxide partial pressure sensor selected from the group consisting of: said arterial blood carbon dioxide partial pressure sensor and said end-tidal exhaled air carbon dioxide partial pressure sensor, to maintain said arterial blood carbon dioxide partial pressure level at least one pressure range selected from the group consisting of 41 to 45 mm Hg, 46 to 50 mm Hg, 51 to 55 mm Hg, 56 to 65 mm Hg.

35. The system as in claim 28, wherein said controllable mixing module controllably mixes carbogen gas of 5% carbon dioxide and 30% molecular oxygen and 65% of chemical element Xenon with a gas mixture of 30% molecular oxygen and 70% of chemical element Xenon according to feedback from said at least one carbon dioxide partial pressure sensor selected from the group consisting of: said arterial blood carbon dioxide partial pressure sensor and said end-tidal exhaled air carbon dioxide partial pressure sensor, to maintain said arterial blood carbon dioxide partial pressure level at 41 to 65 mm Hg.

36. The system as in claim 28, wherein said controllable mixing module controllably mixes carbogen gas of 5% carbon dioxide and 50% molecular oxygen and 45% of chemical element Xenon with a gas mixture of 50% molecular oxygen and 50% of chemical element Xenon according to feedback from said at least one carbon dioxide partial pressure sensor selected from the group consisting of: said arterial blood carbon dioxide partial pressure sensor and said end-tidal exhaled air carbon dioxide partial pressure sensor, to maintain said arterial blood carbon dioxide partial pressure level at 41 to 65 mm Hg.

37. The system as in claim 28, wherein said controllable mixing module controllably mixes carbogen gas of 5% carbon dioxide and 30% molecular oxygen and 65% of chemical element Argon with a gas mixture of 30% molecular oxygen and 70% of chemical element Argon according to feedback from said at least one carbon dioxide partial pressure sensor selected from the group consisting of: said arterial blood carbon dioxide partial pressure sensor and said end-tidal exhaled air carbon dioxide partial pressure sensor, to maintain said arterial blood carbon dioxide partial pressure level at 41 to 65 mm Hg.

38. The system as in claim 28, wherein said controllable mixing module controllably mixes carbogen gas of 5% carbon dioxide and 50% molecular oxygen and 45% of chemical element Argon with a gas mixture of 50% molecular oxygen and 50% of chemical element Argon according to feedback from said at least one carbon dioxide partial pressure sensor selected from the group consisting of: said arterial blood carbon dioxide partial pressure sensor and said end-tidal exhaled air carbon dioxide partial pressure sensor, to maintain said arterial blood carbon dioxide partial pressure level at 41 to 65 mm Hg.

39. A method of a synchronized cardiopulmonary resuscitation (CPR), configured to ensure delivery of an efficient amount of oxygen to essential organs whilst prevent cerebral hypocapnia by maximizing delivery of carbon dioxide to a brain, comprises:
(a) exerting a distal-to-proximal sequential compression force onto a limb by compressing at least one limb device and occluding a blood flow into said limb, wherein said exerting of said compression force is performed constantly and not intermittently;
(b) delivering a mixture of gases by a positive-pressure ventilation comprising:
(I) providing a carbon dioxide enriched gas;
(II) providing a molecular oxygen enriched gas;
(III) controllably mixing said molecular oxygen enriched gas with said carbon dioxide enriched gas;
(IV) detecting a partial pressure of carbon dioxide in an arterial blood;
(V) controlling at least one ratio selected from the group consisting of: a ratio of said molecular oxygen enriched gas and ratio of said carbon dioxide enriched gas, in a mixture of said molecular oxygen enriched gas and said carbon dioxide enriched gas;
wherein said controlling is configured to include an efficient amount of said molecular oxygen in said ratio of said mixture of said molecular oxygen enriched gas and said carbon dioxide enriched gas, and wherein said controlling is further configured to increase an amount of said carbon dioxide enriched gas in said ratio of said mixture of said molecular oxygen enriched gas and said carbon dioxide enriched gas, thereby maximizing delivery of carbon dioxide to a brain and preventing cerebral hypocapnia;
(c) providing an endotracheal tube comprising a unidirectionally sealing cuff disposed at a distal portion of said endotracheal tube;
(d) conferring to said unidirectionally sealing cuff a deployed configuration, wherein said unidirectionally sealing cuff is engaged to an interior surface of a trachea, thereby effectively sealing a passage of gasses in-between said unidirectionally sealing cuff and said interior surface of a trachea, whilst sustaining an inflow of gases from said endotracheal tube, into said trachea;
wherein conferring of said deployed configuration is performed by said unidirectionally sealing cuff during an inhaling phase of a respiratory cycle;
(e) conferring to said unidirectionally sealing cuff a withheld configuration, wherein said unidirectionally sealing cuff is disengaged from said interior surface of said trachea, whilst sustaining a spontaneous outflow of said gases from said trachea;
wherein said conferring of withheld configuration is performed by said unidirectionally sealing cuff during an exhaling phase of said respiratory cycle;
(f) performing a cardiac stimulation to a cardiac muscle, for returning a spontaneous circulation of said arterial blood, by providing at least one type of stimulation selected from the group consisting of: a mechanical stimulation and electrical stimulation, at a predetermined time intervals;
(g) continuously determining a pressure inside said trachea;
(h) synchronizing a timing of an injection phase of said positive-pressure ventilation system with an onset of a decompression phase of said cardiac stimulation.

40. The system as in claim 28, wherein said compressing of at least one limb device and occluding said blood flow into said limb is performed by an up-rolling constricting elastic ring.

41. The system as in claim 28, wherein said compressing of at least one limb device and occluding said blood flow into said limb is performed by applying at least one element selected from the group consisting of: an elastic bandage, an elastic limb wrap with adjustable closures, an inflatable limb wrap with adjustable closures.

42. The system as in claim 28, wherein said at least one limb device is configured for occluding the arterial inflow of blood into the limb by applying a surface skin pressure range selected from the group consisting of: 100 to 200 mm Hg, 200 to 300 mm Hg.

43. An endotracheal device for delivering a semi-spontaneous positive-pressure ventilation comprises:
(a) an elongated tube configured for endotracheal deployment, comprising an interior lumen;
(b) a unidirectionally sealing cuff disposed at a distal portion of said elongated tube, configured for iteratively and intermittently assuming a deployed configuration and withheld configuration;
(c) in said deployed configuration, said unidirectionally sealing cuff is sprawled out, so as to engage to an interior surface of a trachea, thereby effectively sealing a passage of gasses in-between said unidirectionally sealing cuff and said interior surface of a trachea, whilst sustaining an inflow of gases from said endotracheal tube, into said trachea;
(d) in said withheld configuration, said unidirectionally sealing cuff is folded, so as to disengage from said interior surface of said trachea, whilst sustaining a spontaneous outflow of said gases from said trachea;
(e) wherein said deployed configuration is assumable by said unidirectionally sealing cuff during an inhaling phase of a respiratory cycle;
(f) wherein said withheld configuration is assumable by said unidirectionally sealing cuff during an exhaling phase of said respiratory cycle.

44. The endotracheal device, as in claim 43, wherein said sealing cuff comprises an inflatable toroidal structure, comprising an inflatable interior lumen.

45. The endotracheal device, as in claim 44, further comprises at least one conduit connecting said inflatable interior lumen of said sealing cuff with said interior lumen of said elongated tube.

46. The endotracheal device, as in claim 44, further comprises at least one outlet on an anterior distal portion of said toroidal structure of said sealing cuff, configured to sustain an inflow of gases from said inflatable interior lumen of said sealing cuff into said trachea.

47. The endotracheal device, as in claim 43, wherein said elongated tube comprises a unidirectional flow check-valve, configured to sustain an inflow of gases from said endotracheal tube, into said trachea.
